(19) United States
(12) Patent Application Publication
(10) Pub. No.: US 2021/0330834 A1
(43) Pub. Date: Oct. 28, 2021

(54) Hand Hygiene Hub and Method

(71) Applicant: Blisk Corp, Hawkinsville, GA (US)

(72) Inventor: Shalom J Goldberg, Hawinksville, GA (US)

(73) Assignee: Blisk Corp, Hawkinsville, GA (US)

(21) Appl. No.: 17/238,205

(22) Filed: Apr. 23, 2021

Related U.S. Application Data
(60) Provisional application No. 63/014,646, filed on Apr. 23, 2020.

(51) Int. Cl. A61L 2/10; A61L 2/24

(52) U.S. Cl. CPC A61L 2/10; A61L 2/24; A61L 2202/20; A61L 2202/11; A61L 2202/14

(57) ABSTRACT

A method for sterilizing a hand of a subject is disclosed. In this method a hand is inserted into a Hand Hygiene Hub. The presence of the hand and/or a pathogen on the hand is sensed of a subject. In response to the pathogen and/or hand's presence being sensed, the inserted hand is automatically irradiated with 222 nm Ultraviolet-C light within the Hand Hygiene Hub. The hand is irradiated for a predetermined period of time that is set based on a type of the pathogen sensed.

This application claims the benefit of U.S. Provisional Application No. 63/014,646, filed on Apr. 23, 2020 the contents of which are hereby incorporated by record.

### TECHNICAL FIELD

These claimed embodiments relate to a Hand Hygiene Hub for removing pathogens on a subject's hand and more particularly to a method for sterilizing the hands of a subject.

### BACKGROUND OF THE INVENTION

Numerous industries such as the healthcare, foodservice, beer brewers, hospitality, public facilities use medical skin disinfection devices/gels containing a chemical compound to disinfect hands. The users typically wash their hands to remove debris, to remove grease and to remove the dead pathogens, and then finally uses a chemical compound containing at least a 60% ethyl alcohol solution. Maximum full disinfection in hand hygiene is often missing in all of the healthcare environments. Also using chemicals to disinfect hands can be mess, expensive and often results in skin damage ranging to cracked, bleeding palms.

Existing hand safety measures/standards do not ensure that a healthcare provider would actually carry out the hand washing step, because many assume that the alcohol when combined with the gloves is going to be completely adequate. Furthermore, the spread of pathogens would have been traced back to poor hand safety standards if this step is missed, or purposefully skipped. The rushed environment of most healthcare environments is given the priority at times, and it could be that in those hastened times the subject becomes infected due to careless hygiene practices and waving existing protocols.

### SUMMARY OF THE INVENTION

This application relates to a Hand Hygiene Hub (H-3). The Hub may be wall mounted and disinfects and decontaminate Hands. The Hub incorporates LAN (Local Area Network) software to verify, and time stamp a subject's identity and validate their use of the Hub. The Hub delivers safe UV-C light to render a pathogen disinfecting quality by using the specific Care222® Far UV-C Excimer lamps developed by USHIO America, Inc. The UV-C lamps deliver the 222 nm UV-C Wavelength exclusively to achieve pathogen inactivation by relying on the Excimer technology. This device will safely disinfect hands and validate usage by users.

In one implementation, a method for disinfecting the hands of a subject includes inserting one or more body parts in a hand hygiene hub and sensing the presence of a one or more pathogens on a hand of a subject. The inserted hand is automatically irradiated by the Ultraviolet-C light within the Hand Hygiene Hub for a predetermined period of time based on a type of the one or more pathogens sensed.

In another implementation, a hygiene hub that is provided includes a plurality of walls forming a chamber with an opening to receive a hand of a subject. One or more sensors are coupled with the plurality of walls to detect within the chamber the presence of one or more pathogens present on the hand of the subject. An ultraviolet-c light source operative to emit 222 nm ultraviolet (UV-C) light onto the hand of the subject in response to the one or more sensors detecting the presence of one or more pathogens present on the hand of the subject.

### BRIEF DESCRIPTION OF THE DRAWINGS

The detailed description is described with reference to the accompanying figures. In the figures, the left-most digit(s) of a reference number identifies the figure in which the reference number first appears. The use of the same reference number in different figures indicates similar or identical items.

FIG. 1 is an isometric view of the Hand Hygiene Hub;

FIG. 2 is a schematic diagram of the Hand Hygiene Hub;

FIG. 3 is a simplified schematic diagram of an exemplary hosting computing device used in the system for sterilizing a subject's hand; and

FIG. 4 illustrates a flow diagram of a process for using the hub to sterilize a subject's hand.

### DETAILED DESCRIPTION

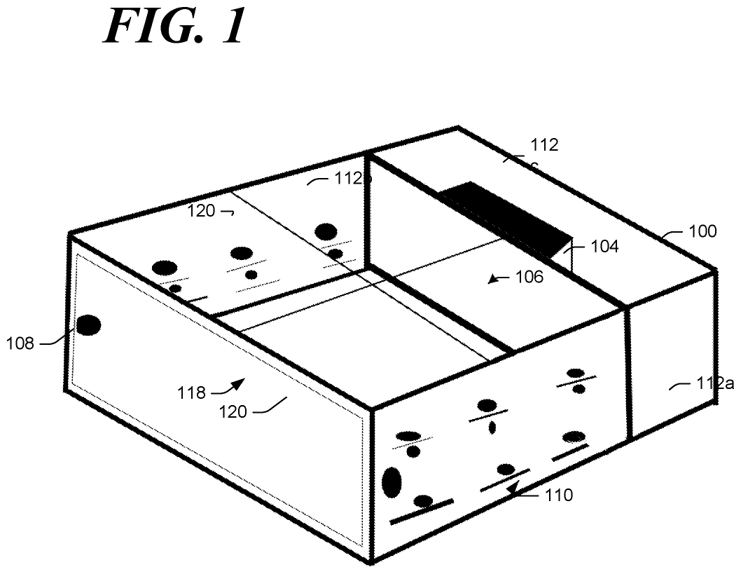

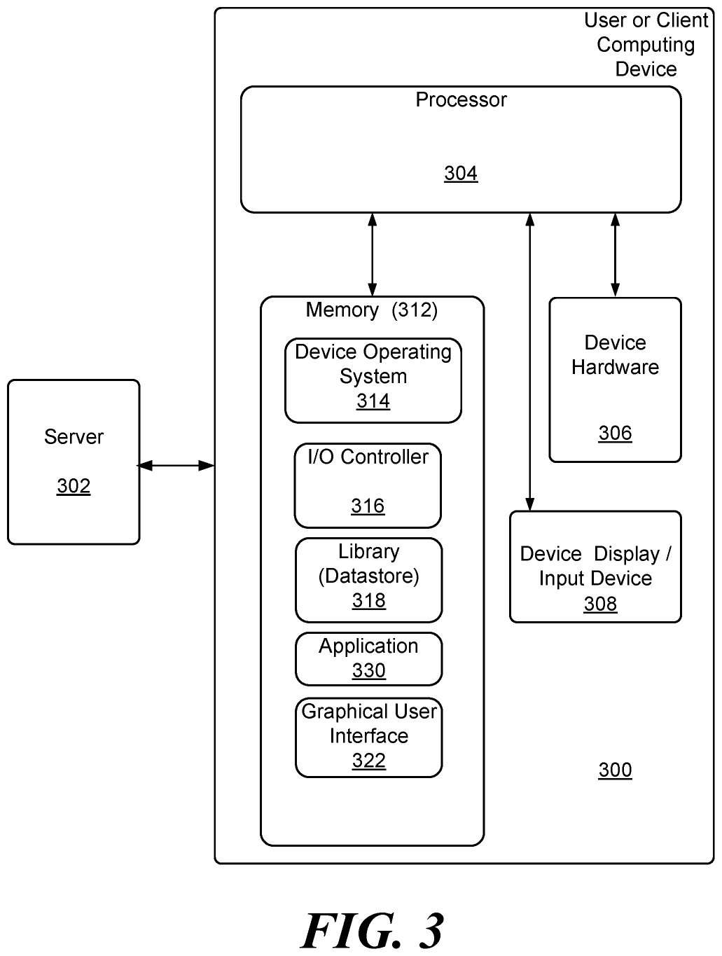

Referring to FIG. 1 there is shown a station 100 for sterilizing a subject's hand. The station 100 is preferably centrally located and is made of ABS plastic. The station 100 includes a computing device 102 (e.g., a tablet) that is described in more detail in connection with FIG. 3. Station 102 also includes a controller 104 coupled with multiple 222 nm ultraviolet (UV-C) bulbs (or lamps) 106, Infrared sensors 108, and subsonic transducers 110.

Station 100, also referred to herein as a Hub, is formed with three side walls 112a-c, a top panel 114 and a bottom panel 116 the combination enclosing an inner chamber 118. A front of station 100 is connected to walls 112a and 112b, cover panel 114 and bottom panel 118. The front has an outer frame 120 forming an opening 122 through which one or more hands may be inserted to inner chamber 118. Bulbs 106 may be Far UV-C Care222® lamps produced by USHIO, Inc and arranged in an array that delivers a singular wavelength of 222 nm.

The subsonic sound emitters are connected to the transducers 110 may be an array of super-sensitive 4 or more piezoelectric chip-based transducers designed to operate in the subsonic range, receiving sound waves from three equal distanced aligned receptors. The receptors for these transducers 110 may be doubled to reinforce their modulation. Transducers 110 are directed at the sides and top and bottom of the hand of the subject during operation and are operable to detect unique fractional sound patterns of various pathogens (pathogen types) present at various spots on the hand.

Transducers 110, IR sensors and UV-C lamps are controlled by controller 104 which receives commands from computing device 102. Infrared (IR) sensors 108 and transducers 110 provide information regarding what was sensed to computing device 102 via controller 104. Computing device 102 may maintain a library of known pathogens types and their respective ultrasonic patterns. Upon receiving a pattern from transducers 110 and an indication of a hand present from the IR sensor 108, computing device may determine the type of pathogen present on the hand by comparing the pattern against known patterns in its library. Computing device 102 may then provide a signal to lamps 106 via controller 104 to turn the UV-C lamps 106 on for a calculated and unique time to kill the pathogen(s). Such calculated time may be set each time anew based on the determined pathogen type. Computing device 102 may also provide an audio indication or an indication on the display indicating the UV-C lamp 106 is on, or that the UV-C lamp 106 is off, and that the pathogen has been killed. The computing device 102 may have the results of the scan posted to its internal storage and may transmits the pathogen results to a hand-held unit that a healthcare provider may carry or a remote database/server.

The UV-C bulbs 106 may be mounted to the cover panel 114 and bottom panel 118 to simultaneously radiate both sides of the hand when inserted. IR sensors 108 and the one or more subsonic transducers 110 may likewise be mounted to the cover panel 114 and bottom panel 118.

Station 100 may form a left slot and a right slot within the chamber 118 to simultaneously receive a left hand and a right hand of the subject. Each side (top and bottom) of each hand slot may contain three UV-C 222 nm lamps triggered to be turned on in response to an indication from the IR (infra-Red) sensor 108 and/or the transducers 110 when the subject's hands are inserted. In one implementation 5 UVC lamps 106 are positioned on top and 5 positioned at the bottom to provide full coverage for each hand, top bottom, and sides.

Station 100 may also include an interior metal skeleton to hold the components, sensors, and lamps 106 in place permanently. Circuitry in controller 104 may be accessed through the rear panel when the cover panel 114 is removed after the entire housing of the Hub 100 is detached from a cover mounting plate 114 connecting the station 100 to the wall. The cover mounting plate 114 may be attached by screws directly to the studs in the walls. The hub 100 could be detachable with a special key that a service technician will carry to remove the hub 100 from its locked state to the wall in order to provide servicing.

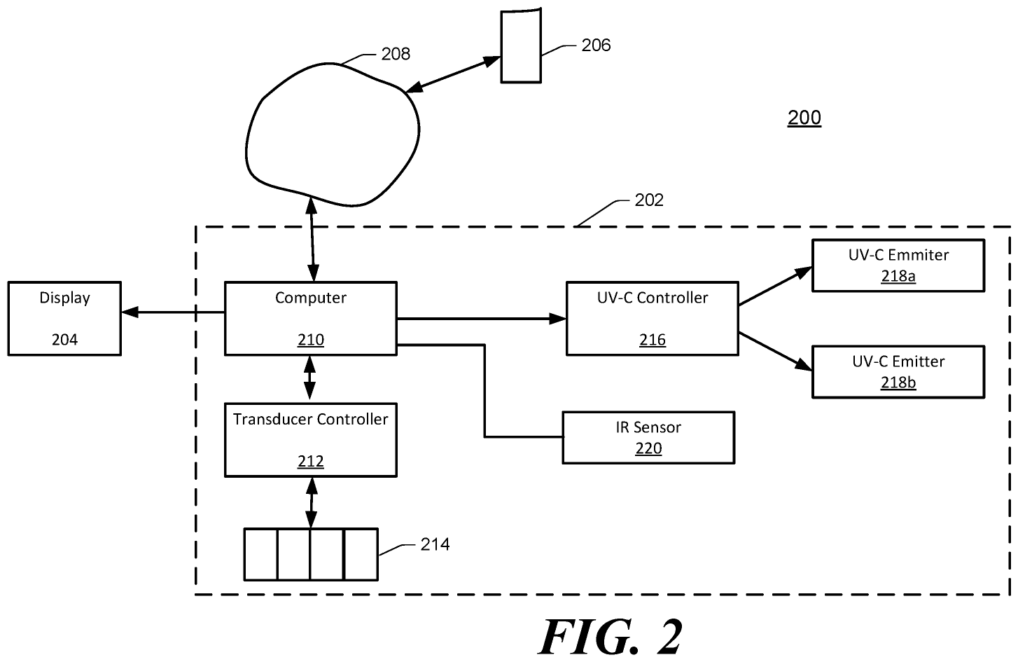

In FIG. 2, there is shown a simplified system diagram 200 of the hand hygiene hub 202 (100 FIG. 1). The Hand hygiene hub 202 may be coupled to a display and user interface device 204 and may be connected to a server/datastore 206 via a network 208 (such as the internet and/or a local area network).

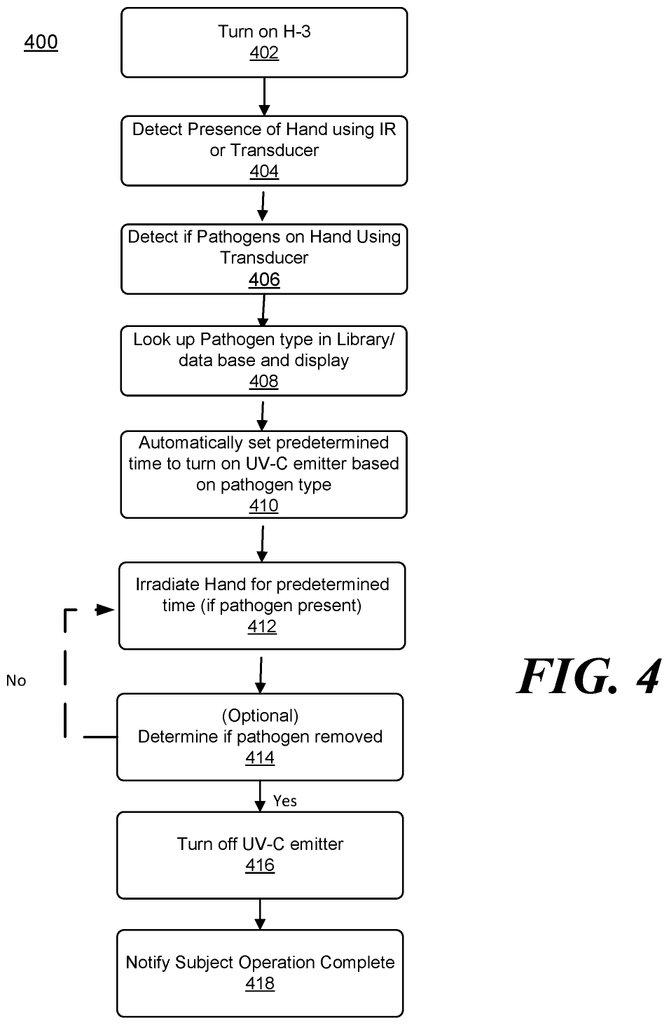

Hand hygiene hub 202 includes a computer device 210 coupled via transducer controller 212 to transducer array 214. Transducer array 214 may include multiple emitter and receptor pairs. Computer device 210 is also coupled via bulb/lamp controller 216 to UV-C bulb array 218. Details of computing device 210 are shown in FIG. 3. Controller 216 may also be coupled via controller 212 or directly to IR sensor 220. Controllers 212 and 216 may include in controller 104 shown in FIG. 1. Operation of the Hygiene hub is described in more detail in FIG. 4.

### Example Architecture

In FIG. 3 there are illustrated selected modules in computing device 300 (Computing Device 102 of FIG. 1 and 210 of FIG. 2) using process 400 shown in FIG. 4. Computing device 300 includes a processing device 304, memory 312, and hardware 306, which is connected to bulb controller 212 and transducer controller 216 (FIG. 2). Processing device 304 may include one or more microprocessors, microcontrollers or any such devices for accessing memory 312 or hardware 306. Processing device 304 has processing capabilities and memory 312 suitable to store and execute computer-executable instructions.

Processing device 304 executes instruction stored in memory 312, and in response thereto, processes signals (e.g., binary logic levels) from hardware 314. Hardware 314 may include a display 334, controller 216 and/or 216, and input device 308 and an I/O device controller 316. I/O device may include a network and communication circuitry for communicating with network 208. Input device 308 receives inputs from a user of the host computing device 300 and may include a keyboard, mouse, track pad, microphone, audio input device, video input device, or touch screen display. Display device (204 FIG. 2) may include an LED, LCD, CRT, or any type of display device.

Memory 312 may include volatile and nonvolatile memory, removable and non-removable media implemented in any method or technology for storage of information, such as computer-readable instructions, data structures, program modules or other data. Such memory includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, RAID storage systems, or any other medium which can be used to store the desired information, and which can be accessed by a computer system.

Stored in memory 312 of the hosting device 300 may include an operating system 314, application 330 and a library 318 of other applications including database 330. Operating system 314 may be used by application 320 to operate device 300. The operating system 314 may include drivers for device 300 to communicate with input device 308 and I/O device. Library 330 may include preconfigured parameters (or set by the user before or after initial operation) such web site operating parameters, web site libraries, HTML (Hyper Text Markup Language) libraries, API's (Application program interface) and configurations.

Stored in the library 318 in memory 312 (or received from external server 302) are the parameters regarding the pathogen patterns corresponding to patterns that may be sensed by transducer array 214 that indicate a pathogen type. Also stored in the library may be predetermined time periods necessary to turn on the lamps to destroy the pathogen type.

Application 320 includes a graphical user interface module 322 for displaying pathogens and subject information.

The exemplary process in FIG. 4 is illustrated as a collection of blocks in a logical flow diagram, which represents a sequence of operations that can be implemented in hardware, software, and a combination thereof. In the context of software, the blocks represent computer-executable instructions that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described blocks can be combined in any order and/or in parallel to implement the process. For discussion purposes, the processes are described with reference to FIG. 4, although it may be implemented in other system architectures.

Referring to FIG. 4, a flowchart of process 400 performed by processor 304 when executing the software instructions in application 330 is shown. Process 400 includes blocks 402-418. When application 330 is executed on the host computing device 300, it uses the processor 304 and instructions in modules 314-322 that are shown in FIG. 3.

In the process 400 in block 402, the computing processor 304 and hub 202 is turned on. When turned on processor 304 runs the operating system and ensures that the IR sensor 220 is turned on.

In block 404, processor 304 receives a signal from an IR controller (not shown) and/or the transducer array 214 via the controller indicating a hand of a subject is present in the chamber 118.

In block 406, the processor 304 activates the transducer array 214 to send a signal via the controller to the hand to detect if one or more pathogens are present. The processor 304 receives a signal via the controller indicating the pattern detected by the transducer array 214. During this same time, the subjects' hands could be scanned for any remaining pathogens and their disinfection could be recorded by the system.

In block 408, the processor 304 obtains a match for the pattern detected in the library/datastore 330 to determine a pathogen type or alternatively requests additional patterns from a remote server/datastore. Stored with the pathogen type is an indication of the predetermined time to irradiate the hand to kill the pathogen.

In block 410, the processor 304 saves a predetermined time to turn on and/or the intensity of the UV-C lamp array 218 based on the pathogen type. The processor 304 may provide an indication to the display indicating the pathogen type and indicate a start of the irradiation by the UV-C lamp array 218.

In block 412, the processor 304 automatically turns on the UV-C lamp array 218 for the predetermined time.

In block 414, the processor 304 may receive a signal from the transducer array 214 indicating if the pathogen pattern is still present. If the pattern is still present block 412 may be repeated.

If the pattern of the pathogens is not present, in block 416 the processor 304 turns off the lamps and may in block 418 provide an indication to the display indicating the lamp array 218 is turned off, the pathogen has been irradicated and that the subject may remove their hand. Processor 304 may also initiate the transmission of disinfection result data and earlier pathogen scans to the other external computing devices.

In one implementation, computer device 300 will be a personal device assistant/tablet-like device. with software and will enable the entire management of the subject's well-being to validate that a subject has been sterilized. The tablet may be connected by Wi-Fi, also enabling remote access by authorized personnel.

While the above detailed description has shown, described and identified several novel features of the invention as applied to a preferred embodiment, it will be understood that various omissions, substitutions and changes in the form and details of the described embodiments may be made by those skilled in the art without departing from the spirit of the invention. Accordingly, the scope of the invention should not be limited to the foregoing discussion but should be defined by the appended claims.

What is claimed is:

1. A method for disinfecting the hands of a subject comprising:
inserting one or more body parts in a hand hygiene hub;
sensing the presence of a one or more pathogens on a hand of a subject; and
automatically irradiating the inserted hand with Ultraviolet light within the hand hygiene hub for a predetermined period of time based on a type of the one or more pathogens sensed.

2. The method as recited in claim 1 further comprising:
identifying the type of the one or more pathogens sensed with a controller using pattern recognition.

3. The method as recited in claim 2 wherein the ultraviolet light is emitted at 222 nm.

4. The method as recited in claim 1, wherein sensing the presence of a one or more pathogens on hand includes using one or more ultrasonic transducers transmitting in the sonic range to sense the presence of the one or more pathogens.

5. The method as recited in claim 1, wherein sensing the presence of a one or more pathogens on the hand includes using multiple ultrasonic transducers transmitting in the sonic range and placed in array pattern to sense the presence of the one or more pathogens.

6. The method as recited in claim 2 further comprising:
transmitting a signal indicating the identified pathogen type to a processing device; and
displaying to a user with the processing device the identified pathogen type.

7. The method as recited in claim 2 wherein automatically irradiating the inserted hand with Ultraviolet light within the hand hygiene hub includes automatically irradiating the inserted hand with multiple ultraviolet light sources within the hand hygiene hub, and placing a least one of the UV light sources below the hand and another of the UV light sources over the hand within the hand hygiene hub.

8. A Hygiene hub comprising:
a plurality of walls forming a chamber with opening to receive a hand of a subject;
one or more sensors coupled with the plurality of walls to detect within the chamber the presence of one or more pathogens present on the hand of the subject; and
an ultraviolet light source operative to emit 222 nm ultraviolet (UV) light onto the hand of the subject in response to the one or more sensors detecting the presence of one or more pathogens present on the hand of the subject.

9. The hub as recited in claim 8, further comprising a controller and an archive library stored in a memory of the hub to identify a type of the one or more pathogens using pattern recognition.

10. The hub as recited in claim 9, further comprising a controller operative to set the time the UV light is emitted based on the pathogen type.

11. The hub as recited in claim 10, wherein the one or more sensors include multiple ultrasonic transducers transmitting in the sonic range and are placed in array pattern to sense the presence of the one or more pathogens.

12. A method for disinfecting one or more body parts of a subject comprising:
placing the one or more body parts adjacent a hygiene device;
sensing, with an ultrasonic transducer coupled with the hygiene device and transmitting in a subsonic range, one or more pathogens on the one or more body parts;
identifying, using a controller coupled with the transducer, a type of the one or more pathogens on the one or more body parts; and
irradiating the one or more body parts with 222 nm Ultraviolet light for a predetermined period of time, the predetermined time set based on the identified type of the one or more pathogens.

13. The method as recited in claim 12 wherein sensing the presence of a one or more pathogens on the one or more body parts includes using multiple ultrasonic transducers transmitting in the sonic range and placed in array pattern to sense the presence of the one or more pathogens.
